(19) United States
(12) Patent Application Publication
(10) Pub. No.: US 2022/0347009 A1
(43) Pub. Date: Nov. 3, 2022

(54) Targeted Temperature Management Systems, Pads, and Methods

(71) Applicant: C. R. Bard, Inc., Franklin Lakes, NJ (US)

(72) Inventors: James David Hughett, SR., Monroe, GA (US); Gabriel A. Johnston, Broomfield, CO (US); Michael R. Hoglund, Windsor, CO (US); Sean E. Walker, Platteville, CO (US); Madeline Stich, Thornton, CO (US); Rebecca D. Salisbury, Lafayette, CO (US)

(21) Appl. No.: 17/723,210

(22) Filed: Apr. 18, 2022

Related U.S. Application Data
(60) Provisional application No. 63/183,452, filed on May 3, 2021.

(51) Int. Cl. A61F 7/00

(52) U.S. Cl. CPC A61F 7/0085

(57) ABSTRACT

Disclosed herein are systems, pads, and methods for targeted temperature management. For example, a pad can include a multilayered pad body, a pad inlet connector, and a pad outlet connector. The pad body can include a conduit layer and a non-adhesive, thermally conductive layer over the conduit layer. The conduit layer can include one or more conduits configured to convey a temperature-controlled fluid as a supply fluid from a hydraulic system of a control module. The one-or-more conduits can also configured to convey the temperature-controlled fluid as a return fluid back to the hydraulic system. The conductive layer can be configured for placement on a portion of a patient's body. The pad inlet connector can include a pad inlet configured for charging the conduit layer with the supply fluid. The pad outlet connector can include a pad outlet configured for discharging the return fluid from the conduit layer.

### PRIORITY

This application claims the benefit of priority to U.S. Provisional Patent Application No. 63/183,452, filed May 3, 2021, which is incorporated by reference in its entirety into this application.

### BACKGROUND

Targeted temperature management (“TTM”) is a treatment for maintaining therapeutic body temperatures (e.g., hypothermia, hyperthermia, etc.) in patients to improve their outcomes in different medical situations. Current systems for TTM generally use adhesive pads placed on different portions of the patient's bodies to circulate temperature-controlled fluid (e.g., cooled fluid or warmed fluid) about the patients for inducing or maintaining therapeutic body temperatures. The adhesive pads are adhered onto the patient's bodies in order to maintain sufficient contact between the adhesive pads and the patient's bodies for the TTM. However, such pads can cause skin irritation from the adhesive of the adhesive pads. What is needed are TTM systems, pads, and methods that reduce or eliminate the skin irritation caused from the adhesive of the adhesive pads for TTM.

Disclosed herein are TTM systems, pads, and methods that address at least the foregoing need.

### SUMMARY

Disclosed herein is a pad for TTM. The pad includes, in some embodiments, a multilayered pad body, a pad inlet connector, and a pad outlet connector. The pad body includes a conduit layer and a non-adhesive, thermally conductive layer over the conduit layer. The conduit layer includes one or more conduits configured to convey a temperature-controlled fluid as a supply fluid from a hydraulic system of a control module. The one-or-more conduits are also configured to convey the temperature-controlled fluid as a return fluid back to the hydraulic system. The conductive layer is configured for placement on a portion of a patient's body. The pad inlet connector includes a pad inlet configured for charging the conduit layer with the supply fluid. The pad outlet connector includes a pad outlet configured for discharging the return fluid from the conduit layer.

In some embodiments, the conductive layer includes a thermally conductive mesh.

In some embodiments, the conductive layer includes a plurality of conductive studs disposed in the conductive layer. The conductive studs are configured to enhance thermal conduction between the temperature-controlled fluid and the patient's body.

In some embodiments, the conductive studs are distributed over the conductive layer in a regular pattern.

In some embodiments, the conductive studs are distributed over the conductive layer in an irregular pattern. The irregular pattern has areas of higher concentrations for greater thermal conduction.

In some embodiments, the conductive studs extend into the one-or-more conduits for direct contact with the temperature-controlled fluid.

In some embodiments, the conductive layer includes a plurality of suction cups integrated into the conductive layer. The suction cups are configured to adhere to the patient's body and secure the pad to the patient's body.

In some embodiments, the suction cups are configured to pull blood to a surface of the patient's body to enhance thermal conduction between the temperature-controlled fluid and the patient's body.

In some embodiments, the pad body further includes a plurality of magnets around a perimeter of the pad body. The magnets include magnetic pairs having opposing magnetic moments located across the pad from each other for securing the pad around the patient's body or the portion thereof.

In some embodiments, the pad further includes an impermeable film between the conduit layer and the conductive layer. The impermeable film is configured to retain the temperature-controlled fluid in the conduit layer.

In some embodiments, the conduit layer includes a plurality of protrusions extending from the conduit layer toward the impermeable film. The protrusions are configured to promote even flow of the temperature-controlled fluid.

In some embodiments, the pad further includes a secondary fluid delivery line (“FDL”). The secondary FDL is configured to convey the supply fluid from the hydraulic system. The secondary FDL is also configured to convey the return fluid back to the hydraulic system. The secondary FDL is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

Also disclosed herein is a system for TTM. The system includes, in some embodiments, a control module, a primary FDL, and one or more pads. The control module includes a hydraulic system. The hydraulic system is configured to provide a temperature-controlled fluid. The primary FDL is configured to convey the temperature-controlled fluid from the hydraulic system as a supply fluid. The FDL is also configured to convey the temperature-controlled fluid back to the hydraulic system as a return fluid. The one-or-more pads are configured for placement on one or more portions of a patient's body, respectively. Each pad of the one-or-more pads includes a multilayered pad body. The pad body includes a conduit layer and a non-adhesive, thermally conductive layer over the conduit layer. The conduit layer includes one or more conduits configured to convey the temperature-controlled fluid. The conductive layer is configured for placement on a portion of a patient's body.

In some embodiments, the conductive layer includes a thermally conductive mesh.

In some embodiments, the conductive layer includes a plurality of conductive studs disposed in the conductive layer. The conductive studs are configured to enhance thermal conduction between the temperature-controlled fluid and the patient's body.

In some embodiments, the conductive studs are distributed over the conductive layer in a regular pattern.

In some embodiments, the conductive studs are distributed over the conductive layer in an irregular pattern. The irregular pattern has areas of higher concentrations for greater thermal conduction.

In some embodiments, the conductive studs extend into the one-or-more conduits for direct contact with the temperature-controlled fluid.

In some embodiments, the conductive layer includes a plurality of suction cups integrated into the conductive layer. The suction cups are configured to adhere to the patient's body and secure the pad to the patient's body.

In some embodiments, the suction cups are configured to pull blood to a surface of the patient's body to enhance thermal conduction between the temperature-controlled fluid and the patient's body.

In some embodiments, the pad body further includes a plurality of magnets around a perimeter of the pad body. The magnets include magnetic pairs having opposing magnetic moments located across the pad from each other for securing the pad around the patient's body or the portion thereof.

In some embodiments, the pad further includes an impermeable film between the conduit layer and the conductive layer. The impermeable film is configured to retain the temperature-controlled fluid in the conduit layer.

In some embodiments, the conduit layer includes a plurality of protrusions extending from the conduit layer toward the impermeable film. The protrusions are configured to promote even flow of the temperature-controlled fluid.

In some embodiments, the pad further includes a secondary FDL. The secondary FDL is configured to convey the supply fluid from the hydraulic system. The secondary FDL is also configured to convey the return fluid back to the hydraulic system. The secondary FDL is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

In some embodiments, the hydraulic system further includes a chiller evaporator, a heater, a hydraulic-system outlet, and a hydraulic-system inlet. The chiller evaporator is configured for fluid cooling. The heater is configured for fluid heating. The chiller evaporator and the heater, together, are configured to provide the temperature-controlled fluid. The hydraulic-system outlet is configured for discharging the supply fluid from the hydraulic system. The hydraulic-system inlet is configured for charging the hydraulic system with the return fluid to continue to produce the temperature-controlled fluid.

In some embodiments, the control module further includes one or more processors, primary memory, and instructions stored in the primary memory. The instructions are configured to instantiate one or more processes for TTM with the control module when executed by the one-or-more processors.

Also disclosed herein is a method of a system for TTM. The method includes, in some embodiments, a pad-placing step, a pad-charging step, and a fluid-circulating step. The pad-placing step includes placing a pad on a portion of a patient's body with a non-adhesive, thermally conductive layer of a multilayered pad body of the pad in contact with skin of the portion of the patient's body. The pad-charging step includes charging one or more conduits of a conduit layer of the pad with a supply fluid of a temperature-controlled fluid. The supply fluid is provided by a hydraulic system of a control module by way of a combination of fluidly connected FDLs. The FDLs include a secondary FDL and a primary FDL. The fluid-circulating step includes circulating the supply fluid through a conduit layer of the pad body to cool or warm the portion of the patient's body as needed in accordance with TTM.

In some embodiments, the conductive layer includes a thermally conductive mesh.

In some embodiments, the conductive layer includes a plurality of conductive studs disposed in the conductive layer. The conductive studs are configured to enhance thermal conduction between the temperature-controlled fluid and the patient's body.

In some embodiments, the conductive studs are distributed over the conductive layer in a regular pattern.

In some embodiments, the conductive studs are distributed over the conductive layer in an irregular pattern. The irregular pattern has areas of higher concentrations for greater thermal conduction.

In some embodiments, the conductive studs extend into the one-or-more conduits for direct contact with the temperature-controlled fluid.

In some embodiments, the method further includes a first pad-securing step. The first pad-securing step includes adhering a plurality of suction cups integrated into the conductive layer to the patient's body to secure the pad to the patient's body.

In some embodiments, the suction cups are configured to pull blood to a surface of the patient's body to promote vasodilation and enhance thermal conduction between the temperature-controlled fluid and the patient's body.

In some embodiments, the method further includes a second pad-securing step. The second pad-securing step includes coupling together magnetic pairs of a plurality magnets around a perimeter of the pad body to secure the pad to the patient's body.

In some embodiments, the method further comprises an FDL-connecting step. The FDL-connecting step includes fluidly connecting a secondary FDL outlet connector at a split pad-connecting end of the secondary FDL to a pad inlet connector. The FDL-connecting step also includes fluidly connecting a secondary FDL inlet connector at the split pad-connecting end of the secondary FDL to a pad outlet connector.

In some embodiments, the fluid-circulating step includes transferring heat between the temperature-controlled fluid and the portion of the patient's body by thermal conduction through the conductive layer.

In some embodiments, the fluid-circulating step includes circulating a cool fluid through the conduit layer to bring the patient into hypothermia from normothermia.

In some embodiments, the fluid-circulating step includes circulating a warm fluid through the conduit layer to bring the patient into normothermia from hypothermia.

In some embodiments, the fluid-circulating step includes circulating a warm fluid through the conduit layer to bring the patient into hyperthermia from normothermia.

In some embodiments, the fluid-circulating step includes circulating a cool fluid through the conduit layer to bring the patient into normothermia from hyperthermia.

These and other features of the concepts provided herein will become more apparent to those of skill in the art in view of the accompanying drawings and following description, which describe particular embodiments of such concepts in greater detail.

### DRAWINGS

FIG. 1 illustrates a system for TTM including a console and one or more pads in accordance with some embodiments.

FIG. 2A illustrates left and right torso pads of the one-or-more pads in accordance with some embodiments.

FIG. 2B illustrates left and right leg pads of the one-or-more pads in accordance with some embodiments.

FIG. 3 illustrates a multilayered pad body of a pad of the one-or-more pads including a conduit layer and a thermally conductive layer in accordance with some embodiments.

FIG. 4 illustrates a thermally conductive mesh for the conductive layer of FIG. 3 in accordance with some embodiments.

FIG. 5A illustrates a plurality of conductive studs disposed in the conductive layer of FIG. 3 in accordance with some embodiments.

FIG. 5B illustrates a cross-sectional view of the conductive studs disposed in the conductive layer of FIG. 3 in accordance with some embodiments.

FIG. 6A illustrates plurality of suction cups integrated into the conductive layer in accordance with some embodiments.

FIG. 6B illustrates a cross-sectional view of the suction cups integrated into the conductive layer of FIG. 3 in accordance with some embodiments.

FIG. 7 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, current systems for TTM generally use adhesive pads placed on different portions of patient's bodies to circulate temperature-controlled fluid (e.g., cooled fluid or warmed fluid) about the patients for inducing or maintaining therapeutic body temperatures. The adhesive pads are adhered onto the patient's bodies in order to maintain sufficient contact between the adhesive pads and the patient's bodies for the TTM. However, such pads can cause skin irritation from the adhesive of the adhesive pads. What is needed are TTM systems, pads, and methods that reduce or eliminate the skin irritation caused from the adhesive of the adhesive pads for TTM.

Disclosed herein are TTM systems, pads, and methods that address at least the foregoing need.

### Systems for TTM

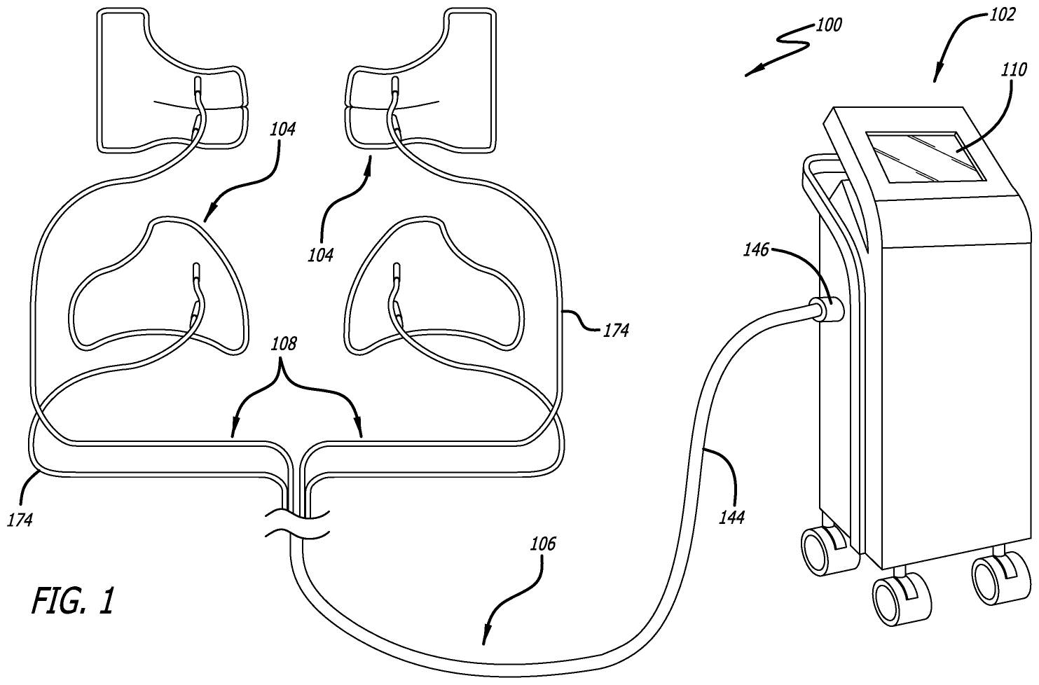

FIG. 1 illustrates a system 100 for TTM including a control module 102 and one or more pads 104 in accordance with some embodiments.

As shown, the system 100 can include the control module 102, the one-or-more pads 104 such as those set forth below, a primary FDL 106, and one or more secondary FDLs 108 corresponding in number to the one-or-more pads 104. Description for the control module 102 is set forth immediately below. Description for the one-or-more pads 104 and the one-or-more secondary FDLs 108 is set forth in the following section.

The control module 102 can include a console 110 with an integrated display screen configured as a touchscreen for operating the control module 102. The control module 102 can include one or more processors, primary and secondary memory, and instructions stored in the primary memory. The instructions are configured to instantiate one or more processes for TTM with the control module 102 when executed by the one-or-more processors.

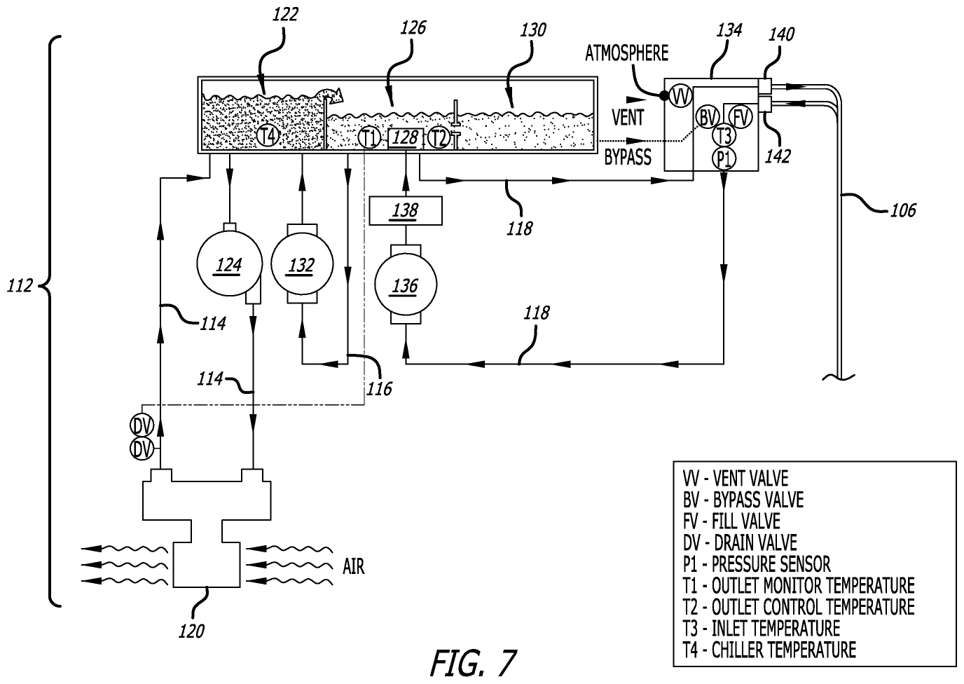

FIG. 7 illustrates a hydraulic system 112 of the control module 102 in accordance with some embodiments.

The control module 102 can also include the hydraulic system 112, which can include a chiller circuit 114, a mixing circuit 116, and a circulating circuit 118 for providing a temperature-controlled fluid.

The chiller circuit 114 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 126 set forth below to produce a supply fluid for TTM. The chiller circuit 114 can include a chiller evaporator 120 configured for the cooling of the fluid passing therethrough. The fluid for the cooling by the chiller evaporator 120 is provided by a chiller tank 122 using a chiller pump 124 of the chiller circuit 114.

The mixing circuit 116 can be configured for mixing spillover of the cooled fluid from the chiller tank 122 with a mixed fluid in a mixing tank 126 of the mixing circuit 116. The mixing circuit 116 can include a heater 128 in the mixing tank 126 configured for heating the mixed fluid to produce a heated fluid, which can be mixed with the cooled fluid in any ratio to provide a supply tank 130 of the circulating circuit 118 with the supply fluid of a desired temperature for TTM. Indeed, the chiller evaporator 120 and the heater 128, together, are configured to cooperate to provide the temperature-controlled fluid. The mixing circuit 116 can include a mixing pump 132 configured to pump the fluid from the mixing tank 126 into the chiller tank 122 for producing the cooled fluid as well as the spillover of the cooled fluid for the mixing tank 126.

The circulating circuit 118 can be configured for circulating the supply fluid for TTM, which includes circulating the supply fluid provided by a manifold 134 through the one-or-more pads 104 using a circulation pump 136 directly or indirectly governed by a flow meter 138 of the circulating circuit 118. The manifold 134 can include an outlet 140 configured for discharging the supply fluid (e.g., a cooled fluid or a warmed fluid as indicated) from the hydraulic system 112 and an inlet 142 configured for charging the hydraulic system 112 with return fluid from the one-or-more pads 104 to continue to produce the supply fluid.

The primary FDL 106 can include primary tubing 144 configured to convey the supply fluid from the hydraulic system 112 by way of a lumen of the primary tubing 144 when fluidly connected to the hydraulic system 112. Likewise, the primary tubing 144 is configured convey the return fluid back to the hydraulic system 112 by way of the lumen of the primary tubing 144 when fluidly connected to the hydraulic system 112.

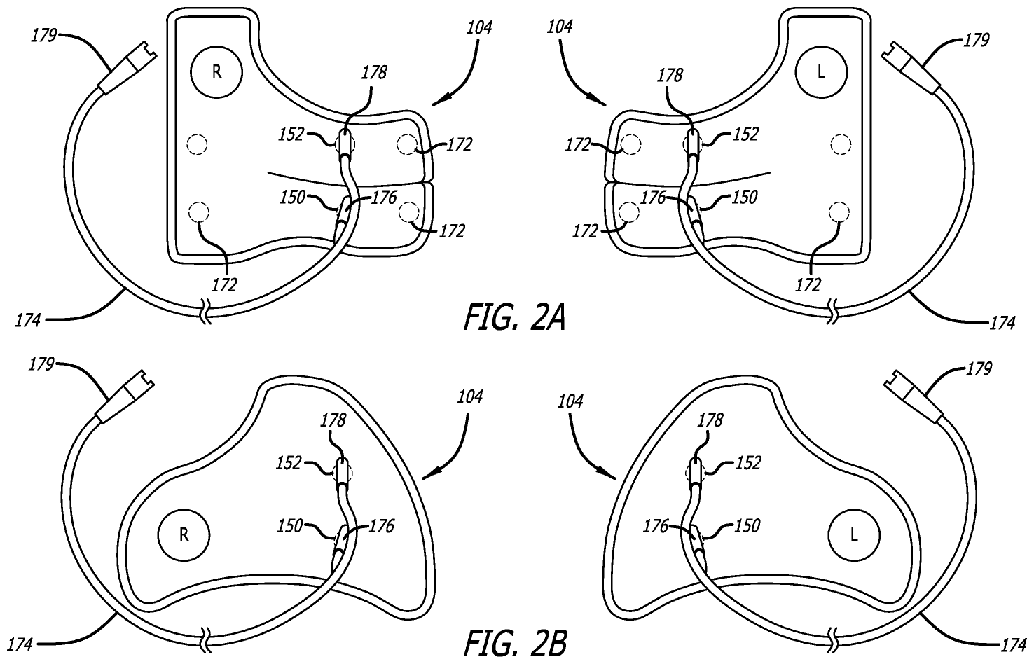

The primary tubing 144 of the primary FDL 106 of FIG. 1 can include a pair of opposing connector ends, wherein each connector end of the pair of connector ends includes a corresponding primary FDL connector. For example, a connector end of the pair of connector ends can include a control module-connecting primary FDL connector 146 configured to fluidly connect to a control-module connector (not shown) of the control module 102. The control-module connector can include both the outlet 140 and the inlet 142 of the hydraulic system 112. Another connector end of the pair of connector ends can include a secondary FDL-connecting primary FDL connector (not shown) configured to fluidly connect to the primary FDL-connecting secondary FDL connector 179 set forth below. (See FIGS. 2A and 2B.) The secondary FDL-connecting primary FDL connector can include both an outlet and an inlet.

### Pads for TTM

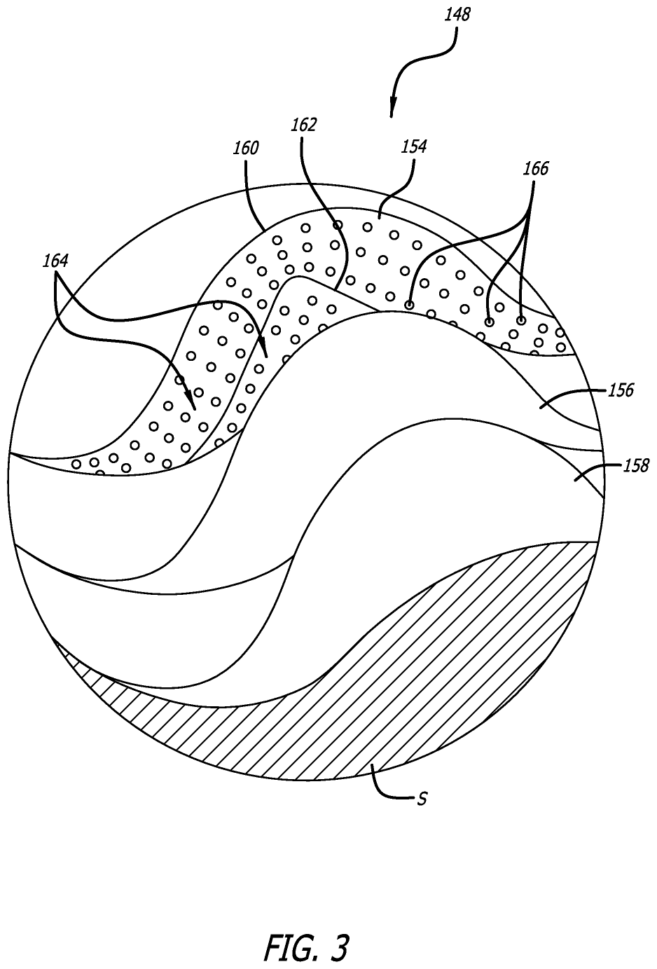

FIGS. 2A and 2B illustrate left and right pads of the one-or-more pads 104 respectively for a torso and legs of a patient in accordance with some embodiments. However, the one-or-more pads 104 can be configured for placement on any one or more portions of a patient's body, respectively, not just the torso or legs. FIG. 3 illustrates a multilayered pad body 148 of a pad of the one-or-more pads 104 in accordance with some embodiments.

A pad of the one-or-more pads 104 can include the pad body 148, a pad inlet connector 150, and a pad outlet connector 152.

The pad body 148 can include a conduit layer 154, an impermeable film 156 over the conduit layer 154, and a non-adhesive, thermally conductive layer 158 over both the conduit layer 154 and the impermeable film 156.

The conduit layer 154 can include a perimetrical wall 160 and one or more inner walls 162 extending from the conduit layer 154 toward the impermeable film 156. Together with the impermeable film 156, the perimetrical wall 160 and the one-or-more inner walls 162 form one or more conduits 164 configured to convey through the conduit layer 154 the temperature-controlled fluid as the supply fluid from the hydraulic system 112 or the return fluid back to the hydraulic system 112.

The conduit layer 154 can include a plurality of protrusions 166 extending from the conduit layer 154 toward the impermeable film 156. The protrusions 166 can be configured to promote even flow of the temperature-controlled fluid as the supply fluid or the return fluid when conveyed through the conduit layer 154.

The conduit layer 154 can be a unitary piece of an opaque polymer (e.g., foam) or a translucent polymer such as an elastomer (e.g., silicone).

The impermeable film 156 can be configured to retain the temperature-controlled fluid as the supply fluid or the return fluid in the conduit layer 154 when the conveyed through the conduit layer 154. In addition, the impermeable film 156 can be configured to allow efficient energy transfer between the conduit layer 154 and the conductive layer 158.

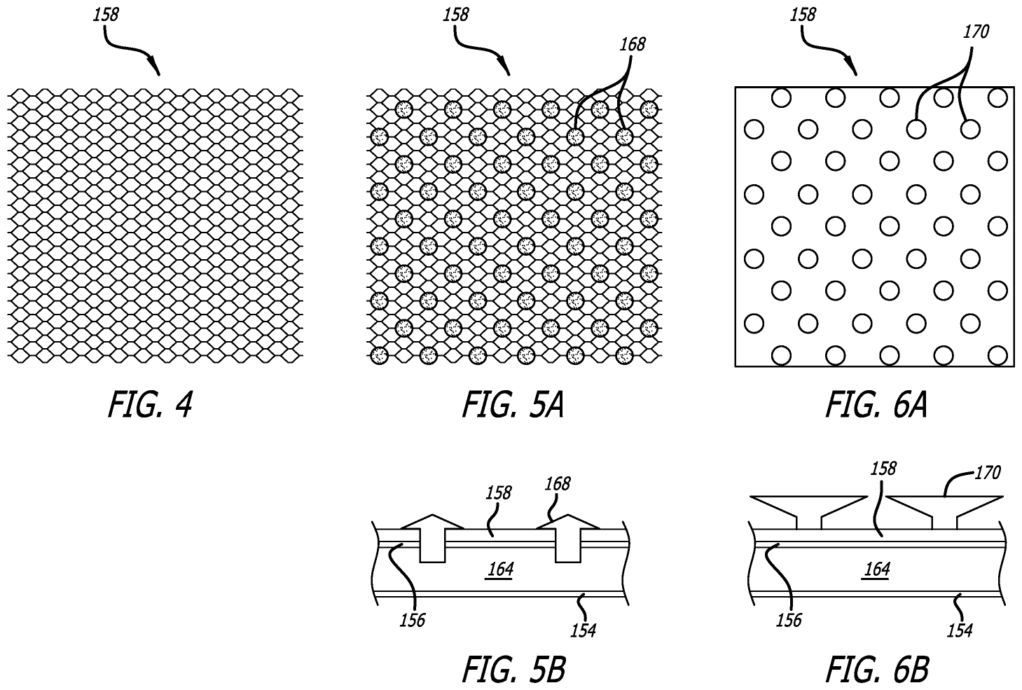

The conductive layer 158 can be configured for placement on skin S (see FIG. 3) of a portion (e.g., torso, leg, etc.) of a patient's body for direct thermal conduction through the conductive layer 158. The conductive layer 158 can be a thermally conductive mesh as shown in FIG. 4.

FIGS. 5A and 5B illustrate a plurality of conductive studs 168 disposed in the conductive layer 158 in accordance with some embodiments.

As shown, the conductive layer 158 can include the conductive studs 168 disposed in the conductive layer 158 for enhancing thermal conduction between the temperature-controlled fluid and a patient's body. Indeed, the conductive studs 168 can extend from the pad body 148 into the one-or-more conduits 164 for direct contact with the temperature-controlled fluid as shown in FIG. 5B. The conductive studs 168 can be distributed over the conductive layer 158 in a regular pattern or an irregular pattern as needed for enhancing thermal conduction between the temperature-controlled fluid and the patient's body. For example, the irregular pattern can have areas of higher concentrations of the conductive studs 168 corresponding to more blood-rich portions of the patient's body requiring greater thermal conduction. Likewise, the irregular pattern can have areas of lower concentrations of the conductive studs 168 corresponding to less blood-rich portions of the patient's body requiring less thermal conduction.

FIGS. 6A and 6B illustrate a plurality of suction cups 170 disposed in the conductive layer 158 in accordance with some embodiments.

Additionally or alternatively, the conductive layer 158 can include the suction cups 170 (e.g., micro-suction cups) integrated into the conductive layer 158. The suction cups 170 can be configured to adhere to a patient's body and secure the pad to the patient's body. The suction cups 170 can also be configured to pull blood to a surface of the patient's body to enhance thermal conduction between the temperature-controlled fluid and the patient's body. While such a configuration can include static suction cups as the suction cups 170, the configuration can alternatively include dynamic suction cups fluidly connected to a pump of the control module 102 through one or more conduits in the conductive layer 158 or another conduit layer between the conduit layer 154 and the conductive layer 158. Advantageously, the pump can be used to draw a baseline vacuum to secure the pad to the patient's body and, periodically, draw a greater-than-baseline vacuum to promote vasodilation to counter any vasocontraction caused by cooling the patient. For further effect, the greater-then-baseline vacuum can be drawn alternately with cooling the patient.

Whether or not the conductive layer 158 includes the suction cups 170, the pad body 148 can include a plurality of magnets 172 around a perimeter of the pad body 148. The magnets 172 can include magnetic pairs having opposing magnetic moments located across the pad from each other for securing the pad around a patient's body or the portion thereof. The magnets 172, suction cups 170, or both, advantageously allow for quickly placing and securing the one-or-more pads 104. Indeed, the magnets 172, suction cups 170, or both allow for more quickly placing and securing the one-or-more pads 104 over, for example, securing straps.

The pad inlet connector 150 can be configured for charging the conduit layer 154 with the supply fluid, while the pad outlet connector 152 can be configured for discharging the return fluid from the conduit layer 154.

A pad of the one-or-more pads 104 can include a secondary FDL of the one-or-more secondary FDLs 108. For example, the secondary FDL can be pre-connected to the pad as sold.

The secondary FDL can include secondary tubing 174 configured to convey the supply fluid from the primary FDL 106 when connected to the hydraulic system 112 of the control module 102. Likewise, the secondary tubing 174 can be configured to convey the return fluid back to the primary FDL 106 when connected to the hydraulic system 112 of the control module 102.

The secondary FDL can be split at a pad-connecting end of the secondary FDL. The pad-connecting end of the secondary FDL can include a pair of pad-connecting secondary FDL connectors. A pad-connecting secondary FDL inlet connector 176 of the pair of pad-connecting secondary FDL connectors can be configured to fluidly connect to the pad outlet connector 152. A pad-connecting secondary FDL outlet connector 178 of the pair of pad-connecting secondary FDL connectors can be configured to fluidly connect to the pad inlet connector 150.

The secondary FDL need not be split at a primary FDL-connecting end of the secondary FDL like the pad-connecting end of the secondary FDL. Indeed, an unsplit primary FDL-connecting end of the secondary FDL facilitates quickly connecting the one-or-more secondary FDLs 108 to the primary FDL 106. Accordingly, the primary FDL-connecting end of the secondary FDL can include a single primary FDL-connecting secondary FDL connector 179 configured to fluidly connect to the secondary FDL-connecting primary FDL connector (not shown) set forth above. The primary FDL-connecting secondary FDL connector 179 can include both an inlet and an outlet corresponding to the outlet and the inlet of the secondary FDL-connecting primary FDL connector.

### Methods

Methods of the system 100 or the one-or-more pads 104 include methods of use. For example, a method of using the system 100 for TTM can include one or more steps selected from a pad-placing step, a pad-securing step, an FDL-connecting step, a pad-charging step, and a fluid-circulating step.

The pad-placing step can include placing a pad of the one-or-more pads 104 on a portion of a patient's body with the non-adhesive, thermally conductive layer 158 of the pad body 148 in contact with skin S (see FIG. 3) of the portion of the patient's body.

If the pad body 148 includes the suction cups 170 integrated into the conductive layer 158 of the pad body 148, the magnets 172 around the perimeter of the pad body 148, or both, the method can include the pad-securing step. The pad-securing step can include adhering the suction cups 170 to a patient's body to secure the pad to the patient's body. Additionally or alternatively, the pad-securing step can include coupling together the magnetic pairs of the magnets 172 to secure the pad to the patient's body.

If a secondary FDL of the one-or-more secondary FDLs 108 is not connected to the foregoing pad, the method can include the FDL-connecting step. The FDL-connecting step can include fluidly connecting the pad-connecting secondary FDL outlet connector 178 at the split pad-connecting end of the secondary FDL to the pad inlet connector 150. The FDL-connecting step can also include fluidly connecting the pad-connecting secondary FDL inlet connector 176 at the split pad-connecting end of the secondary FDL to the pad outlet connector 152.

The pad-charging step can include charging the one-or-more conduits 164 of the conduit layer 154 of the pad with the supply fluid. As set forth above, the supply fluid can be provided by the hydraulic system 112 of the control module 102 by way of a combination of fluidly connected FDLs including the secondary FDL and the primary FDL 106.

The fluid-circulating step can include circulating the supply fluid through the conduit layer 154 of the pad body 148 to cool or warm a portion of a patient's body as needed in accordance with TTM. Indeed, the fluid-circulating step can include transferring heat between the supply fluid and the portion of the patient's body by thermal conduction through the conductive layer 158. For example, the fluid-circulating step can include circulating a cool fluid through the conduit layer 154 to bring the patient into hypothermia from normothermia. In another example, the fluid-circulating step can include circulating a warm fluid through the conduit layer 154 to bring the patient into normothermia from hypothermia. In yet another example, the fluid-circulating step can include circulating a warm fluid through the conduit layer 154 to bring the patient into hyperthermia from normothermia. In yet another example, the fluid-circulating step can include circulating a cool fluid through the conduit layer 154 to bring the patient into normothermia from hyperthermia.

While the method set forth above is described with reference to a single pad of the one-or-more pads 104, it should be understood that any number of pads of the one-or-more pads 104 can be used as necessary to effectuate a desired treatment by way of the system 100 for TTM.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A pad for targeted temperature management (“TTM”), comprising:
a multilayered pad body including:
a conduit layer including one or more conduits configured to convey a temperature-controlled fluid as a supply fluid from a hydraulic system of a control module and convey a return fluid back to the hydraulic system; and
a non-adhesive, thermally conductive layer over the conduit layer configured for placement on a portion of a patient's body;
a pad inlet connector including a pad inlet configured for charging the conduit layer with the supply fluid; and
a pad outlet connector including a pad outlet configured for discharging the return fluid from the conduit layer.

2. The pad of claim 1, wherein the conductive layer includes a thermally conductive mesh.

3. The pad of claim 1, wherein the conductive layer includes a plurality of conductive studs disposed in the conductive layer, the conductive studs configured to enhance thermal conduction between the temperature-controlled fluid and the patient's body.

4. The pad of claim 3, wherein the conductive studs are distributed over the conductive layer in a regular pattern.

5. The pad of claim 3, wherein the conductive studs are distributed over the conductive layer in an irregular pattern, the irregular pattern having areas of higher concentrations for greater thermal conduction.

6. The pad of claim 3, wherein the conductive studs extend into the one-or-more conduits for direct contact with the temperature-controlled fluid.

7. The pad of claim 1, wherein the conductive layer includes a plurality of suction cups integrated into the conductive layer, the suction cups configured to adhere to the patient's body and secure the pad to the patient's body.

8. The pad of claim 7, wherein the suction cups are configured to pull blood to a surface of the patient's body to enhance thermal conduction between the temperature-controlled fluid and the patient's body.

9. The pad of claim 1, the pad body further comprising a plurality of magnets around a perimeter of the pad body, the magnets including magnetic pairs having opposing magnetic moments located across the pad from each other for securing the pad around the patient's body or the portion thereof.

10. The pad of claim 1, further comprising an impermeable film between the conduit layer and the conductive layer configured to retain the temperature-controlled fluid in the conduit layer.

11. The pad of claim 10, wherein the conduit layer includes a plurality of protrusions extending from the conduit layer toward the impermeable film, the protrusions configured to promote even flow of the temperature-controlled fluid.

12. The pad of claim 1, further comprising:
a secondary fluid delivery line (“FDL”) configured to convey the supply fluid from the hydraulic system and convey the return fluid back to the hydraulic system, the secondary FDL split at a pad-connecting end of the secondary FDL, and the pad-connecting end of the secondary FDL including a pair of secondary FDL connectors including a secondary FDL outlet connector configured to fluidly connect to the pad inlet connector and a secondary FDL inlet connector configured to fluidly connect to the pad outlet connector.

13. A system for targeted temperature management (“TTM”), comprising:
a control module including a hydraulic system configured to provide a temperature-controlled fluid;
a primary fluid delivery line (“FDL”) configured to convey the temperature-controlled fluid from the hydraulic system as a supply fluid and convey the temperature-controlled fluid back to the hydraulic system as a return fluid; and
one or more pads configured for placement on one or more portions of a patient's body, respectively, each pad of the one-or-more pads including a multilayered pad body including:
a conduit layer including one or more conduits configured to convey the temperature-controlled fluid; and
a non-adhesive, thermally conductive layer over the conduit layer configured for placement on the patient's body.

14. The system of claim 13, wherein the conductive layer includes a thermally conductive mesh.

15. The system of claim 13, wherein the conductive layer includes a plurality of conductive studs disposed in the conductive layer, the conductive studs configured to enhance thermal conduction between the temperature-controlled fluid and the patient's body.

16. The system of claim 15, wherein the conductive studs are distributed over the conductive layer in a regular pattern.

17. The system of claim 15, wherein the conductive studs are distributed over the conductive layer in an irregular pattern, the irregular pattern having areas of higher concentrations for greater thermal conduction.

18. The system of claim 15, wherein the conductive studs extend into the one-or-more conduits for direct contact with the temperature-controlled fluid.

19. The system of claim 13, wherein the conductive layer includes a plurality of suction cups integrated into the conductive layer, the suction cups configured to adhere to the patient's body and secure the pad to the patient's body.

20. The system of claim 19, wherein the suction cups are configured to pull blood to a surface of the patient's body to enhance thermal conduction between the temperature-controlled fluid and the patient's body.

21. The system of claim 13, the pad body further comprising a plurality of magnets around a perimeter of the pad body, the magnets including magnetic pairs having opposing magnetic moments located across the pad from each other for securing the pad around the patient's body or the portion thereof.

22. The system of claim 13, further comprising an impermeable film between the conduit layer and the conductive layer configured to retain the temperature-controlled fluid in the conduit layer.

23. The system of claim 22, wherein the conduit layer includes a plurality of protrusions extending from the conduit layer toward the impermeable film, the protrusions configured to promote even flow of the temperature-controlled fluid.

24. The system of claim 13, further comprising:
a secondary fluid delivery line (“FDL”) configured to convey the supply fluid from the hydraulic system and convey the return fluid back to the hydraulic system, the secondary FDL split at a pad-connecting end of the secondary FDL, and the pad-connecting end of the secondary FDL including a pair of secondary FDL connectors including a secondary FDL outlet connector configured to fluidly connect to a pad inlet connector and a secondary FDL inlet connector configured to fluidly connect to a pad outlet connector.

25. The system of claim 13, the hydraulic system further including:
a chiller evaporator configured for fluid cooling;
a heater configured for fluid heating, the chiller evaporator and the heater, together, configured to provide the temperature-controlled fluid;
a hydraulic-system outlet configured for discharging the supply fluid from the hydraulic system; and
a hydraulic-system inlet configured for charging the hydraulic system with the return fluid to continue to produce the temperature-controlled fluid.

26. The system of claim 13, the control module further including: one or more processors, primary memory, and instructions stored in the primary memory configured to instantiate one or more processes for TTM with the control module.

27-41. (canceled)
